(19) United States
(12) Patent Application Publication
(10) Pub. No.: US 2010/0168259 A1
(43) Pub. Date: Jul. 1, 2010

(54) PROMOTED CARBIDE-BASED FISCHER-TROPSCH CATALYST, METHOD FOR ITS PREPARATION AND USES THEREOF

(76) Inventors: Tiancun Xiao, Oxford (GB); Yangdong Qian, Buckinghamshire (GB)

Correspondence Address: RENNER OTTO BOISSELLE & SKLAR, LLP, 1621 EUCLID AVENUE, NINETEENTH FLOOR, CLEVELAND, OH, 44115, US

(21) Appl. No.: 12/528,824

(22) PCT Filed: Feb. 29, 2008
(86) PCT No.: PCT/GB2008/000703

§ 371 Date: Feb. 1, 2010

(30) Foreign Application Priority Data
Mar. 1, 2007 (GB) 0704003.3

(51) Int. Cl. C07C 27/06; B01J 21/08; B01J 23/75; B01J 23/89; B01J 23/46

(52) U.S. Cl. 518/719; 502/242; 502/332; 502/327; 518/715

(57) ABSTRACT

The present invention is directed towards a precursor for a Fischer-Tropsch catalyst comprising a catalyst support, cobalt or iron on the catalyst support and one or more noble metals on the catalyst support, wherein the cobalt or iron is at least partially in the form of its carbide in the as-prepared catalyst precursor, a method for preparing said precursor and the use of said precursor in a Fischer-Tropsch process.

The present invention relates to a promoted carbide-based Fischer-Tropsch catalyst, a method for its preparation and uses thereof.

Conversion of natural gas to liquid hydrocarbons by a Gas to Liquids (GTL) process or conversion of coal to liquid hydrocarbons by a Coal to Liquids (CTL) process creates a clean, high-performance, liquid fuel which can be used as an alternative to petroleum-based fuels. GTL and CTL processes consist of the three steps of: (1) synthesis gas production; (2) synthesis gas conversion by the Fischer-Tropsch process; and (3) upgrading of Fischer-Tropsch products to desired fuels.

In the Fischer-Tropsch process, a synthesis gas (“syngas”) comprising carbon monoxide and hydrogen is converted in the presence of a Fischer-Tropsch catalyst to liquid hydrocarbons. This conversion step is the heart of the process. The Fischer-Tropsch reaction can be expressed in simplified form as follows:

CO+2H₂→—CH₂—+H₂O.

There have been many patent applications which describe the preparation of Fischer-Tropsch catalysts and processes and reactors for GTL and CTL processes.

There are two primary types of Fischer-Tropsch catalyst: one is iron-based and the other is cobalt-based. There have been many patent applications which describe the preparation of cobalt-based catalysts for Fischer-Tropsch synthesis.

It is also well known that the activity of cobalt-based Fischer-Tropsch catalysts can be improved by the use of promoters and/or modifiers.

Known promoters include those based on alkaline earth metals, such as magnesium, calcium, barium and/or strontium.

Known modifiers include those based on rare earth metals, such as lanthanum or cerium, or d-block transition elements such as phosphorus, boron, gallium, germanium, arsenic and/or antimony.

In an active catalyst, the primary catalyst metal, the promoter(s) and/or the modifier(s) may be present in elemental form, in oxide form, in the form of an alloy with one or more of the other elements and/or as a mixture of two or more of these forms.

Cobalt-based catalysts are generally produced by depositing a cobalt precursor and precursors of any promoters or modifiers onto a catalyst support, drying the catalyst support on which the precursors are deposited and calcining the dried support to convert the precursors to oxides. The catalyst is then generally activated using hydrogen to convert cobalt oxide at least partly into cobalt metal and, if present, the promoter and modifier oxides into the active promoter(s) and modifier(s).

A number of methods are known for producing a catalyst support onto which have been deposited the required precursors.

For instance, WO 01/96017 describes a process in which the catalyst support is impregnated with an aqueous solution or suspension of the precursors of the catalytically active components.

EP-A-0 569 624 describes a process in which the precursors are deposited onto the catalyst support by precipitation.

A further method of depositing precursors onto a catalyst support is the sol-gel method. In the sol-gel method, a metal compound or oxide is hydrolysed in the presence of a stabiliser, such as an amphiphilic betaine, to produce colloidal particles of an oxide. The particles are often co-precipitated onto a support formed from gel precursors of, for example, hydrolysed Si(OMe)₄. An example of such a process is described in DE-A-19 85 2547.

WO 03/0022552 descries an improved cobalt-based Fischer-Tropsch catalyst. In the improved catalyst, the cobalt is present in the catalyst, at least in part, as its carbide. WO 03/002252 also describes methods for the production of such cobalt carbide-based catalysts.

WO 2004/000456 describes improved methods for the production of metal carbide-based catalysts. It is indicated that V, Cr, Mn, Fe, Co, Ni, Cu, Mo and/or W may be used as the primary catalyst metal.

WO 2004/000456 also discloses the use of promoters based on Zr, U, Ti, Th, Ha, Ce, La Y, Mg, Ca, Sr, Cs, Ru, Mo, W, Cr, Mn and/or a rare earth element in connection with cobalt and/or nickel-based catalysts.

The Fischer-Tropsch synthesis is used to produce hydrocarbons. These can range from methane (the C₁ hydrocarbon) to approximately C₅₀ hydrocarbons. Depending on the use to which the hydrocarbons are to be put, it is desirable to be able to obtain hydrocarbons of a suitable size. For instance, for the production of liquid fuels, it is desirable to produce hydrocarbons which predominantly have 5 or more carbon atoms.

It is an aim of the present invention to provide a Fischer-Tropsch catalyst precursor which can be activated to produce a Fischer-Tropsch catalyst which has improved selectivity for the production of hydrocarbons having 5 or more carbon atoms.

It is a further aim of the present invention to provide a Fischer-Tropsch catalyst precursor which can be activated to produce a Fischer-Tropsch catalyst with enhanced activity.

It has also been observed that, if the processes disclosed in the prior art are used to produce Fischer-Tropsch catalyst precursors, there is a tendency to decrease the strength of the support, especially where the catalyst support is shaped to fit into a reactor or is in the form of pellets.

It is a further aim of the present invention to provide a method for producing a Fischer-Tropsch catalyst precursor which reduces the tendency of the catalyst support to decrease in strength.

Therefore, according to a first aspect of the present invention there is provided a precursor for a Fischer-Tropsch catalyst comprising:
(i) a catalyst support;
(ii) cobalt or iron on the catalyst support; and
(iii) one or more noble metals on the catalyst support,
wherein the cobalt or iron is at least partially in the form of its carbide in the as-prepared catalyst precursor.

The cobalt or iron may also be present partially as its oxide or as elemental metal.

Preferably, the catalyst support is a refractory solid oxide, carbon, a zeolite, boronitride or silicon carbide. A mixture of these catalyst supports may be used. Preferred refractory solid oxides are alumina, silica, titania, zirconia and zinc oxide. In particular, a mixture of refractory solid oxides may be used.

If silica is used in the catalyst support for a cobalt-based catalyst, it is preferred that the surface of the silica is coated with a non-silicon oxide refractory solid oxide, in particular zirconia, alumina or titania, to prevent or at least slow down the formation of cobalt-silicate.

The catalyst support may be in the form of a structured shape, pellets or a powder.

Preferably, the catalyst precursor comprises from 10 to 50% cobalt and/or iron (based on the weight of the metal as a percentage of the total weight of the catalyst precursor). More preferably, the catalyst precursor comprises from 15 to 35% of cobalt and/or iron. Most preferably, the catalyst precursor comprises about 30% of cobalt and/or iron.

The catalyst precursor may comprise both cobalt and iron but preferably, the catalyst precursor does not comprise iron.

Preferably, the noble metal is one or more of Pd, Pt, Rh, Ru, Ir, Au, Ag and Os. More preferably, the noble metal is Ru.

It is preferred that the catalyst precursor comprises from 0.01 to 30% in total of noble metal(s) (based on the total weight of all noble metals present as a percentage of the total weight of the catalyst precursor). More preferably, the catalyst precursor comprises from 0.05 to 20% in total of noble metal(s). Most preferably, the catalyst precursor comprises from 0.1 to 5% in total of noble metal(s). Advantageously, the catalyst precursor comprises about 0.2% in total of noble metal(s).

If desired, the catalyst precursor may include one or more other metal-based components as promoters or modifiers. These metal-based components may also be present in the catalyst precursor at least partially as carbides, oxides or elemental metals.

A preferred metal for the one or more other metal-based components is one or more of Zr, Ti, V, Cr, Mn, Ni, Cu, Zn, Nb, Mo, Tc, Cd, Hf, Ta, W, Re, Hg, Tl and the 4f-block lanthanides. Preferred 4f-block lanthanides are La, Ce, Pr, Nd, Pm, Sm, Eu, Gd, Tb, Dy, Ho, Er, Tm, Yb and Lu.

Preferably, the metal for the one or more other metal-based components is one or more of Zn, Cu, Mn, Mo and W.

Preferably, the catalyst precursor comprises from 0.01 to 10% in total of other metal(s) (based on the total weight of all the other metals as a percentage of the total weight of the catalyst precursor). More preferably, the catalyst precursor comprises from 0.1 to 5% in total of other metals. Most preferably, the catalyst precursor comprises about 3% in total of other metals.

Preferably, the catalyst precursor contains from 0.0001 to 10% carbon (based on the weight of the carbon, in whatever form, in the catalyst as percentage of the total weight of the catalyst precursor). More preferably, the catalyst precursor contains from 0.001 to 5% of carbon. Most preferably, the catalyst precursor contains about 0.01% of carbon.

Optionally, the catalyst precursor may contain a nitrogen-containing organic compound such as urea, or an organic ligand such as ammonia or a carboxylic acid, for example acetic acid, which may be in the form of a salt or an ester.

The precursor may be activated to produce a Fischer-Tropsch catalyst, for instance by heating the catalyst precursor in hydrogen and/or a hydrocarbon gas to convert at least some of the carbides to elemental metal.

The present invention also includes the activated catalyst. In the active catalyst, the cobalt or iron is at least partially in the form of its carbide.

Once activated, the catalyst according to this aspect of the present invention has the advantages that it has improved selectivity in a Fischer-Tropsch synthesis for the production of hydrocarbons having five or more carbon atoms. Moreover, especially when Ru is the noble metal, the activity of the catalyst is enhanced.

The catalyst precursor of the first aspect of the present invention may be prepared by any of the methods known in the prior art, such as the impregnation method, the precipitation method or the sol-gel method. However, preferably, the catalyst precursor is prepared by a method of the type described in WO 03/002252 or WO 2004/000456. In any preparation process, it should be ensured that the catalyst support has deposited on it a compound or solvent which enables cobalt or iron carbide to be formed during calcination.

More preferably, the catalyst precursor of the first aspect of the present invention is prepared by use of the method of the second aspect of the present invention described below.

According to a second aspect of the present invention, there is provided a method of preparing a catalyst precursor comprising:
depositing a solution or suspension comprising at least one catalyst metal precursor and a polar organic compound onto a catalyst support, wherein the solution or suspension contains little or no water;
if necessary, drying the catalyst support onto which the solution or suspension has been deposited; and
calcining the catalyst support onto which the solution or suspension has been deposited in an atmosphere containing little or no oxygen to convert at least part of said catalyst metal precursor to its carbide.

The solution or suspension may be applied to the catalyst support by spraying, impregnating or dipping.

Preferably, the solution or suspension contains no water at all, in which case there in no need for the drying step and the calcination step can be carried out directly after the deposition step. However, if a catalyst metal precursor which is a hydrate is used, the solution or suspension will necessarily contain some water of hydration. This water may be sufficient to dissolve some of the components of the solution or suspension, such as urea. However, in some cases, it may be necessary to add some water to the solution or suspension in order to ensure that the catalyst metal precursor(s) and any other components are able to dissolve or become suspended. In such cases, the amount of water used should preferably be the minimum required to allow the catalyst metal precursor(s) and the other components to dissolve or be suspended.

If the solution or suspension contains water, it is preferred that it contains no more than 10%, preferably no more than 5%, most preferably no more than 2% and advantageously no more than 1% by weight of the solution or suspension of water.

Preferably, in the calcination step, the atmosphere contains no oxygen. If the atmosphere contains any oxygen, at least part of the polar organic compound will be oxidised and the oxidised part of the polar organic compound will be unavailable for the formation of carbides.

It is possible to use an atmosphere containing some oxygen. However, in such cases, the level of oxygen present should not be so high as to prevent the formation of a significant amount of metal carbide(s) during the calcination step.

The polar organic compound may be a single polar organic compound or may comprise a mixture of two or more organic compounds, at least one of which is polar.

The polar organic compound(s) is (are) preferably liquid at room temperature (20° C.). However, it is also possible to use polar organic compounds which become liquid at temperatures above room temperature. In such cases, the polar organic compound(s) should preferably be liquid at a temperature below the temperature at which any of the components of the solution or suspension decompose.

Alternatively, the polar organic compound(s) may be selected so that it/they become solubilised or suspended by one or more of the other components used to prepare the solution or suspension. The compound(s) may also become solubilised or suspended by thermal treatment.

Examples of suitable organic compounds for inclusion in the solution or suspension are organic amines, organic carboxylic acids and salts thereof, ammonium salts, alcohols, phenoxides, in particular ammonium phenoxides, alkoxides, in particular ammonium alkoxides, amino acids, compounds containing functional groups such as one or more hydroxyl, amine, amide, carboxylic acid, ester, aldehyde, ketone, imine or imide groups, such as urea, hydroxyamines, trimethylamine, triethylamine, tetramethylamine chloride and tetraethylamine chloride, and surfactants.

Preferred alcohols are those containing from 1 to 30 carbon atoms, preferably 1 to 15 carbon atoms. Examples of suitable alcohols include methanol, ethanol and glycol.

Preferred carboxylic acids are citric acid, oxalic acid and EDTA.

Preferably, the solution or suspension contains a cobalt-containing or an iron-containing precursor. More preferably, the solution or suspension contains a cobalt-containing precursor.

Suitable cobalt-containing precursors include cobalt benzoylacetonate, cobalt carbonate, cobalt cyanide, cobalt hydroxide, cobalt oxalate, cobalt oxide, cobalt nitrate, cobalt acetate, cobalt acetylyactonate and cobalt carbonyl. These cobalt precursors can be used individually or can be used in combination. These cobalt precursors may be in the form of hydrates but are preferably in anhydrous form. In some cases, where the cobalt precursor is not soluble in water, such as cobalt carbonate or cobalt hydroxide, a small amount of nitric acid or a carboxylic acid may be added to enable the precursor to fully dissolve in the solution or suspension.

The solution or suspension may contain at least one primary catalyst metal precursor, such as a cobalt-containing precursor or a mixture of cobalt-containing precursors, and at least one secondary catalyst metal precursor. Such secondary catalyst metal precursor(s) may be present to provide a promoter and/or modifier in the catalyst. Suitable secondary catalyst metals include noble metals, such as Pd, Pt, Rh, Ru, Ir, Au, Ag and Os, transition metals, such as Zr, Ti, V, Cr, Mn, Fe, Co, Ni, Cu, Zn, Nb, Mo, Tc, Cd, Hf, Ta, W, Re, Hg and Ti and the 4f-block lanthanides, such as La, Ce, Pr, Nd, Pm, Sm, Eu, Gd, Tb, Dy, Ho, Er, Tm, Yb and Lu.

Preferred secondary catalyst metals are Pd, Pt, Ru, Ni, Co (if not the primary catalyst metal), Fe (if not the primary catalyst metal), Cu, Mn, Mo and W.

Preferably, the deposition, drying and calcination steps are repeated one or more times. For each repeat, the solution or suspension used in the deposition step may be the same or different.

If the solution or suspension in each repetition is the same, the repetition of the steps allows the amount of catalyst metal(s) to be brought up to the desired level on the catalyst support stepwise in each repetition.

If the solution or suspension in each repetition is different, the repetition of the steps allows schemes for bringing the amounts of different catalyst metals up to the desired level in a series of steps to be executed.

For instance, when the steps are first carried out, the process may lead to the catalyst support having on it all the finally desired amount of the primary catalyst metal. In the following repetition, a secondary metal may be loaded onto the catalyst support. Alternatively, a number of secondary metals may be loaded onto the catalyst support in the first repetition.

Three illustrative schemes for loading metals AA, BB and CC onto a catalyst support are shown below. Numerous other schemes for loading catalyst metals onto a catalyst support will be apparent to a person skilled in the art.

### Schemes for Loading Metals

| | 1 | 2 | 3 |
|---|---|---|---|
| FIRST | AA | ½AA + ½BB | ⅓AA + ⅓BB + ⅓CC |
| PASS | | | |
| FIRST | BB | ½AA + ½BB | ⅓AA + ⅓BB + ⅓CC |
| REPETITION | | | |
| SECOND | CC | CC | ⅓AA + ⅓BB + ⅓CC |
| REPETITION | | | |

Preferably, the catalyst support onto which the solution or suspension has been deposited, if necessary after drying, is calcined using a programmed heating regime which increases the temperature gradually so as to control gas and heat generation from the catalyst metal precursors and the other components of the solution or suspension.

Preferably, during the process, the catalyst support reaches a maximum temperature of no more than 1000° C., more preferably no more than 700° C. and most preferably no more than 500° C. at atmospheric pressure.

The temperature preferably rises at a rate of from 0.0001 to 10° C. per minute, more preferably from 0.1 to 5° C. per minute.

An illustrative programmed heating regime consists of:
(a) maintaining the catalyst support onto which the solution or suspension has been deposited at about room temperature (20° C.) for from 0 to 100, preferably 1 to 20, hours;
(b) heating it to a temperature of from 80 to 120° C., preferably about 100° C.;
(c) maintaining it at the temperature attained in step (b) for at least 10, and preferably at least 15, hours;
(d) heating it at a rate of 0.1 to 10, preferably 0.5 to 5, ° C. per minute to a temperature of from 250 to 800° C., preferably 350 to 400° C.; and
(e) maintaining it at the temperature attained in step (d) for at least 0.1, preferably at least 2 hours.

Optionally, between steps (c) and (d), the catalyst support is heated to a temperature of from 100 to 150° C., maintained at that temperature for from 1 to 10, preferably 3 to 4 hours, heated to about 200° C. and maintained at that temperature for from 1 to 10 hours, preferably 3 to 4 hours.

The drying step, if used, and the calcination step can be carried out in a rotating kiln, in a static oven or in a fluidised bed.

Alternatively, once the calcination step has been completed, either after the steps are first carried out or at the end of a repetition, further catalyst metals may be loaded onto the catalyst support using any of the methods known in the art, in particular any of those described in WO 03/002252 or WO 2004/000456.

The catalyst support may be any one of the catalyst supports conventionally used in the art and in particular may be any one of the catalyst supports mentioned above in connection with the first aspect of the invention.

The method of the second aspect of the invention, especially when the catalyst metals are loaded onto the catalyst support using one or more repetitions of the steps, has been found to be very advantageous because it leads to less destruction of the catalyst support, especially when the catalyst support is in the form of a shaped structure or pellets.

The catalyst precursor of the first aspect of the present invention or the catalyst precursor produced by the method of the second aspect of the invention may be activated by any of the conventional activation processes.

Preferably the catalyst precursor is activated using a reducing gas, such as hydrogen, a gaseous hydrocarbon, a mixture of hydrogen and a gaseous hydrocarbon, a mixture of gaseous hydrocarbons, a mixture of hydrogen and gaseous hydrocarbons or syngas.

The gas may be at a pressure of from 1 bar (atmospheric pressure) to 100 bar and is preferably at a pressure of less than 30 bar.

The catalyst precursor is preferably heated to its activation temperature at a rate of from 0.01 to 20° C. per minute. The activation temperature is preferably no more than 600° C. and is more preferably no more than 400° C.

Preferably, the catalyst precursor is held at the activation temperature for from 2 to 24 hours, more preferably from 8 to 12 hours.

After activation, the catalyst is preferably cooled to the desired reaction temperature.

The catalyst, after activation, is preferably used in a Fischer-Tropsch process. This process may be carried out in a fixed bed reactor, a continuous stirred tank reactor, a slurry bubble column reactor or a circulating fluidized bed reactor.

The Fischer-Tropsch process is well known and the reaction conditions can be any of those known to the person skilled in the art, for instance the conditions described in WO 03/002252 and WO 2004.000456. For example the Fischer-Tropsch process may be carried out at a temperature of from 150 to 300° C., preferably from 200 to 260° C., a pressure of from 1 to 100 bar, preferably from 15 to 25 bar, a H₂ to CO molar ratio of from 1:2 to 8:1, preferably about 2:1, and a gaseous hourly space velocity of from 200 to 5000, preferably from 1000 to 2000.

The present invention is now described, by way of illustration only, in the following Examples. It will be understood that these Example are not limiting and that variations and modifications may be made within the spirit and scope of the invention as set out above and as defined in the following claims.

### EXAMPLE 1

### 10 Wt % Co, 1 Wt % Zr on SiO₂ Catalyst Precursor

A shaped SiO₂ support was raised to a temperature of 450° C. at a rate of 2° C./min and was maintained at this temperature for 10 h prior to its impregnation. At room temperature, 10 g Co(NO₃)₂.6H₂O was mixed with 3-4 g urea in a small beaker. 0.7 g ZrO(NO₃)₂ was dissolved completely with deionised (DI) water (the amount of DI water was determined according to pore volume or H₂O adsorption of the support) in another small beaker. The solution or suspension of ZrO(NO₃)₂ was added to the mixture of Co(NO₃)₂.6H₂O with urea. A clear solution or suspension of ZrO(NO₃)₂, Co(NO₃)₂.6H₂O and urea was obtained after warming. The solution or suspension was added to 13 g of the support (SiO₂) by the incipient wetness impregnation method and dried at about 100° C. in an oven for 12 h. The impregnated catalyst support was subjected to temperature-programmed calcination (TPC) in a static air environment as follows: heated to 130° C. at 1° C./min; maintained at this temperature for 3 h; heated to 150° C. at 0.5° C./min; maintained at this temperature for 3 h; heated to 350° C. at 0.5-1° C./min; and maintained at this temperature for 3 h. Shaped 10% Co, 1% Zr on SiO₂ catalyst precursor was obtained.

### EXAMPLE 2

### 20 Wt % Co, 2 Wt % Zr on SiO₂ Catalyst Precursor

This was prepared as in Example 1, except that the 13 g SiO₂ support was replaced by the 10 wt % Co, 1 wt % Zr on SiO₂ catalyst precursor produced in Example 1.

### EXAMPLE 3

### 30 Wt % Co, 3 Wt % Zr on SiO₂ Catalyst Precursor

This was prepared as in Example 1, except that the 13 g SiO₂ support was replaced by the 20 wt % Co, 2 wt % Zr on SiO₂ catalyst precursor produced in Example 2.

### EXAMPLE 4

### 10 Wt % Co, 1 Wt % Zr on Al₂O₃ Catalyst Precursor

This was prepared as in Example 1, except that 13 g SiO₂ support was replaced by 13 g of Al₂O₃.

### EXAMPLE 5

### 20 Wt % Co, 2 Wt % Zr on Al₂O₃ Catalyst Precursor

This was prepared as in Example 4, except that the 13 g of Al₂O₃ support was replaced by the 10 wt % Co, 1 wt % Zr on Al₂O₃ catalyst precursor produced in Example 4.

### EXAMPLE 6

### 30 Wt % Co, 3 Wt % Zr on Al₂O₃ Catalyst Precursor

This was prepared as in Example 4, except that the 13 g of Al₂O₃ support was replaced by the 20 wt % Co, 2 wt % Zr on Al₂O₃ catalyst precursor produced in Example 5.

### EXAMPLE 7

### 30 Wt % Co, 3 Wt % Zr, 0.5 Wt % Ru on SiO₂ Catalyst Precursor

This was prepared as in Example 3, except that the solution or suspension of ZrO(NO₃)₂, Co(NO₃)₂6H₂O and urea was replaced by 6.7 g of 1.5 wt % Ru(NO)(NO₃)₃ in 5 ml DI H₂O.

### EXAMPLE 8

### 30 Wt % Co, 3 Wt % Zr, 0.1 Wt % Ru on SiO₂ Catalyst Precursor

This was prepared as in Example 7, except that 6.7 g of 1.5 wt % Ru(NO)(NO₃)₃ was replaced by 1.3 g of 1.5 wt % Ru(NO)(NO₃)₃.

### EXAMPLES 9 AND 10

### 30 Wt % Co, 3 Wt % Zr, 0.5 Wt % Ru on Al₂O₃ and 30 Wt % Co, 3 Wt % Zr, 0.1 Wt % Ru on Al₂O₃ Catalyst Precursors

These were prepared as in Examples 7 and 8, except that the SiO₂ was replaced by Al₂O₃.

### EXAMPLE 11

### Co, Zr, Ru on SiO₂ and Co, Zr, Ru on Al₂O₃ Catalyst Precursors

These were prepared as in Example 1-6, except that the solution or suspension of ZrO(NO₃)₂, Co(NO₃)₂.6H₂O and urea was replaced by ZrO(NO₃)₂, Co(NO₃)₂.6H₂O, Ru(NO)(NO₃)₃ and urea.

During the processes set forth in the Examples, there was very little damage to the catalyst support, even when high loading of metals were achieved following a number of repetitions of the steps.

The catalyst precursors produced according to Examples 1 to 11 were activated by flowing H₂ at GHSV of 2000H⁻¹ at a heating rate of 1° C./min to 300° C., maintained at 300° C. for 2 hours and then cooled down to 200° C., at which temperature the reaction is started.

The activated catalysts were used in a Fischer-Tropsch process using the following conditions:

T: 220° C., P: 17.5 bar, GHSV: 2000H⁻¹, H₂/CO ratio: 2.

The results of the Fischer-Tropsch processes are shown in the Table below.

| | TABLE | | | |
|---|---|---|---|---|
| | Catalyst | | | |
| | 30% Co3% Zr/SiO₂ | 30% Co3% Zr0.1% Ru/SiO₂ | 30% Co3% Zr0.5% Ru/SiO₂ | 30% Co3% Zr1% Ru/SiO₂ |
| CO conversion | 50-60% | 68% | 83% | 84% |
| C⁵⁺ | 40-48% | 54% | 66% | 67% |
| productivity | | | | |

As can be seen from the results given in the Table above, use of an activated catalyst according to the invention in a Fischer-Tropsch synthesis leads to greater selectivity for hydrocarbons having five or more carbon atoms and enhanced activity.

### EXAMPLE 12

### 13 Wt % Co, 1.3 Wt % Zr on SiO₂ Catalyst Precursor

A shaped SiO₂ support was raised to a temperature of 450° C. at a rate of 2° C./min and was maintained at this temperature for 10 h prior to its impregnation. At room temperature, 10 g Co(NO₃)₂.6H₂O was mixed with 3-4 g urea in a small beaker. 0.7 g ZrO(NO₃)₂ was dissolved completely with deionised (DI) water (the amount of DI water was determined according to pore volume or H₂O adsorption of the support) in another small beaker. The solution or suspension of ZrO(NO₃)₂ was added to the mixture of Co(NO₃)₂.6H₂O with urea. A clear solution or suspension of ZrO(NO₃)₂, Co(NO₃)₂.6H₂O and urea was obtained after warming. The solution or suspension was added to 13 g of the support (SiO₂) by the incipient wetness impregnation method and dried at about 100° C. in an oven for 12 h. The impregnated catalyst support was subjected to temperature-programmed calcination (TPC) in a static air environment as follows: heated to 130° C. at 1° C./min; maintained at this temperature for 3 h; heated to 150° C. at 0.5° C./min; maintained at this temperature for 3 h; heated to 350° C. at 0.5-1° C./min; and maintained at this temperature for 3 h. Shaped 13% Co, 1.3% Zr on SiO₂ catalyst precursor was obtained.

### EXAMPLE 13

### 22.7 Wt % Co, 2.3 Wt % Zr on SiO₂ Catalyst Precursor

This was prepared as in Example 12, except that 13 g SiO₂ support was replaced by a 13 wt % Co, 1.3 wt % Zr on SiO₂ catalyst precursor of the type produced in Example 12.

### EXAMPLE 14

### 30 Wt % Co, 3.1 Wt % Zr on SiO₂ Catalyst Precursor

This was prepared as in Example 12, except that 13 g SiO₂ support was replaced by a 22.7 wt % Co, 2.3 wt % Zr on SiO₂ catalyst precursor of the type produced in Example 13.

### EXAMPLE 15

### 13 Wt % Co, 1.3 Wt % Zr on Al₂O₃ Catalyst Precursor

This was prepared as in Example 12, except that 13 g SiO₂ support was replaced by 13 g of Al₂O₃.

### EXAMPLE 16

### 22.7 Wt % Co, 2.3 Wt % Zr on Al₂O₃ Catalyst Precursor

This was prepared as in Example 15, except that the 13 g of Al₂O₃ support was replaced by a 13 wt % Co, 1.3 wt % Zr on Al₂O₃ catalyst precursor of the type produced in Example 15.

### EXAMPLE 17

### 30 Wt % Co, 3.1 Wt % Zr on Al₂O₃ Catalyst Precursor

This was prepared as in Example 15, except that the 13 g of Al₂O₃ support was replaced by a 22.7 wt % Co, 2.3 wt % Zr on Al₂O₃ catalyst precursor of the type produced in Example 16.

### EXAMPLE 18

### 30 Wt % Co, 3.1 Wt % Zr, 0.5 Wt % Ru on SiO₂ Catalyst Precursor

This catalyst was prepared according to Example 14. In the preparation, a specific amount of 30 wt % Co, 3.1 wt % Zr on SiO₂ (oxide form after 350° C. calcination) was impregnated with a mixture of 6.7 g of 1.5 wt % Ru(NO)(NO₃)₃ and 5 ml DI H₂O. After impregnation, it was 100° C. in an oven for 12 h. The impregnated catalyst support was subjected to temperature-programmed calcination (TPC) in a static air environment as follows: heated to 130° C. at 1° C./min; maintained at this temperature for 3 h; heated to 150° C. at 0.5° C./min; maintained at this temperature for 3 h; heated to 350° C. at 0.5-1° C./min; and maintained at this temperature for 3 h. A catalyst precursor containing 30 wt % Co, 3.1 wt % Zr, 0.5 wt % Ru on SiO₂ was thus obtained.

### EXAMPLE 19

### 30 Wt % Co, 3.1 Wt % Zr, 0.1 Wt % Ru on SiO₂ Catalyst

This was prepared as in Example 18, except that 6.7 g of 1.5 wt % Ru(NO)(NO₃)₃ was replaced by 1.3 g of 1.5 wt % Ru(NO)(NO₃)₃.

### EXAMPLES 20 AND 21

### 30 Wt % Co, 3.1 Wt % Zr, 0.5 Wt % Ru on Al₂O and 30 Wt % Co, 3.1 Wt % Zr, 0.1 Wt % Ru on Al₂O₃ Catalyst Precursors

These were prepared as in Examples 18 and 19, except that the SiO₂ was replaced by Al₂O₃.

### EXAMPLE 22

### 30% Co3.1% Zr1% Ru/SiO₂ Preparation

This was prepared as in Example 18, except that 6.7 g of 1.5 wt % Ru(NO)(NO₃)₃ was replaced by 13 g of 1.5 wt % Ru(NO)(NO₃)₃.

### Co, Zr, Ru on SiO, and Co, Zr, Ru on Al₂O₃ Catalyst Precursors

These were prepared as in Example 12-17, except that the solution or suspension of ZrO(NO₃)₂, Co(NO₃)₂.6H₂O and urea was replaced by ZrO(NO₃)₂, Co(NO₃)₂.6H₂O, Ru(NO)(NO₃)₃ and urea.

During the processes set forth in the Examples, there was very little damage to the catalyst support, even when high loading of metals were achieved following a number of repeats of the steps.

The catalyst precursors produced according to Examples 12 to 22 were activated by flowing H₂ at GHSV of 2000H⁻¹ at a heating rate of 1° C./min to 300° C., maintained at 300° C. for 2 hours and then cooled down to 200° C., at which temperature the reaction is started.

The activated catalysts were used in a Fischer-Tropsch process using the following conditions:

T: 220° C., P: 17.5 bar, GHSV: 2000H⁻¹, H₂/CO ratio: 2.

The results of the Fischer-Tropsch processes are shown in the Table below.

| | TABLE | | | |
|---|---|---|---|---|
| | Catalyst | | | |
| | 30% Co3.1% Zr/SiO₂ | 30% Co3.1% Zr0.1% Ru/SiO₂ | 30% Co3.1% Zr0.5% Ru/SiO₂ | 30% Co3.1% Zr1% Ru/SiO₂ |
| CO conversion | 50-60% | 68% | 83% | 84% |
| C⁵⁺ | 40-48% | 54% | 66% | 67% |
| productivity | | | | |

As can be seen from the results given in the Table above, use of an activated catalyst according to the invention in a Fischer-Tropsch synthesis leads to greater selectivity for hydrocarbons having five or more carbon atoms and enhanced activity.

### EXAMPLE 23

### Modification of the Silica Support with Titanium: TiO₂/SiO₂

At room temperature, 2.75 g of (C₃H₇O)₄Ti is mixed with 5.95 g of absolute ethanol in a small beaker: the volume of ethanol is determined according to the pore volume of the support. The solution is added to 9.30 g of silica support (sieved between 200-350 micron) by incipient wetness impregnation method. The impregnated support is dried at 100° C. over a hot plate for 3 hours and subjected to temperature-programmed calcination in a muffle furnace, as follows: the sample is introduced at 100° C. in the furnace, the temperature is maintained at 100° C. for 3 hours, the temperature is raised to 350° C. at 2° C./min, the temperature is maintained to 350° C. during 4 hours. A silica titanium modified support is obtained.

### EXAMPLE 24

### First Impregnation with Co

At room temperature, 11.27 g of Co(NO₃)₂.6H₂O is mixed with 4.50 g of urea in a small beaker until a pink paste is obtained. 0.77 g of Zr(NO₃)₂ is mixed with 5.05 g of deionised water (the amount of water is determined by the pore volume of the support obtained in Example 23) and heated over a hot plate at 100° C. until a clear solution is obtained. The solution of Zr(NO₃)₂ is added over the mixture of Co(NO₃)₂.6H₂O and urea. The resulting mixture is heated over a hot plate at 100° C. until a clear red solution is obtained. This solution is added to the support synthesized in Example 23 by incipient wetness impregnation method. The impregnated catalyst is dried over a hot plate at 100° C. for 3 hours and subjected to temperature-programmed calcination in a muffle furnace, as follows: the sample is introduced at 100° C. in the furnace, the temperature is maintained at 100° C. for 3 hours, the temperature is raised to 128° C. at 1° C./min., the temperature is maintained to 128° C. for 3 hours, the temperature is raised to 150° C. at 1° C./min., the temperature is maintained to 150° C. for 3 hours, the temperature is raised to 350° C. at 0.5° C./min., the temperature is maintained to 350° C. for 3 hours. A cobalt impregnated catalyst is obtained.

### EXAMPLE 25

### Second Impregnation with Co to Obtain 30.0% Cu3.0% Zr/5.0% TiO₂/SiO₂

This is prepared as in Example 24 except that the silica titanium modified support of Example 23 is replaced by the cobalt impregnated catalyst obtained in Example 24.

### EXAMPLE 26

### Impregnation with Ru to Obtain 30.0% Cu3.0% Zr/5.0% TiO₂/0.2% Ru/SiO₂

At room temperature, 2 g of Ru(NO)(NO₃)₃ (1.5% Ru in water) is mixed with 4.52 g of water in a small beaker (the amount of water is determined by the pore volume of the catalyst obtained in Example 25). This solution is added to 15 g of the catalyst synthesized in Example 25 by incipient wetness impregnation method. The impregnated support is dried at 100° C. over a hot plate for 3 hours and subjected to temperature-programmed calcination in a muffle furnace, as follows: the sample is introduced at 100° C. in the furnace, the temperature is maintained at 100° C. for 3 hours, the temperature is raised to 350° C. at 2° C./min, the temperature is maintained to 350° C. for 3 hours.

### EXAMPLE 27

### Third Impregnation with Co to Obtain 37.5% Co2.7% Zr/4.5% TiO₂/SiO₂

At room temperature, 9.0 g of Co(NO₃)₂.6H₂O is mixed with 3.6 g of urea in a small beaker until a pink paste is obtained. 4.52 g of deionised water (the amount of water is determined by the pore volume of the catalyst synthesized in Example 25) is heated over a hot plate at 100° C. for 10 min. The hot water is added over the mixture of Co(NO₃)₂.6H₂O and urea. The resulting mixture is heated over a hot plate at 100° C. until a clear red solution is obtained. This solution is added to 15 g of the catalyst synthesized in Example 25 by incipient wetness impregnation method. The impregnated catalyst is dried over a hot plate at 100° C. for 3 hours and subjected to temperature-programmed calcination in a muffle furnace, as follows: the sample is introduced at 100° C. in the furnace, the temperature is maintained at 100° C. for 3 hours, the temperature is raised to 128° C. at 1° C./min., the temperature is maintained to 128° C. for 3 hours, the temperature is raised to 150° C. at 1° C./min., the temperature is maintained to 150° C. for 3 hours, the temperature is raised to 350° C. at 0.5° C./min., the temperature is maintained to 350° C. for 3 hours. A cobalt impregnated catalyst is obtained.

### EXAMPLE 28

### Impregnation with Ru to Obtain 37.5% Co2.7% Zr/4.5% TiO₂/0.2% Ru/SiO₂

This is prepared as in Example 26 except that the cobalt impregnated catalyst obtained in Example 25 is replaced by 15 g of the cobalt impregnated catalyst obtained in Example 27.

### EXAMPLE 29

### Fourth Impregnation with Co to Obtain 44.4% Co2.4% Zr/4.0% TiO₂/SiO₂

This is prepared as in Example 27 except that the cobalt impregnated catalyst obtained in Example 25 is replaced by 14.5 g of the cobalt impregnated catalyst obtained in Example 27.

### EXAMPLE 30

### Impregnation with Ru to Obtain 44.4% Co2.4% Zr/4.0% TiO₂/0.2% Ru/SiO₂

This is prepared as in Example 26 except that the cobalt impregnated catalyst obtained in Example 25 is replaced by 15 g of the cobalt impregnated catalyst obtained in Example 29.

### EXAMPLE 31

### Fifth Impregnation with Co to Obtain 50.9% Co2.1% Zr/3.5% TiO₂/SiO₂

This is prepared as in Example 27 except that the cobalt impregnated catalyst obtained in Example 25 is replaced by 13.7 g of the cobalt impregnated catalyst obtained in Example 29.

### EXAMPLE 32

### Impregnation with Ru to Obtain 50.8% Co2.1% Zr/3.5% TiO₂/0.2% Ru/SiO₂

This is prepared as in Example 26 except that the cobalt impregnated catalyst obtained in Example 25 is replaced by 15 g of the cobalt impregnated catalyst obtained in Example 31.

### Catalytic Results

The catalyst precursors produced according to Examples 25, 27, 28 and 29 were activated in flowing hydrogen at GHSV of 6,000H⁻¹ at the heating rate of 1K/min. to 400° C., and kept for 2 hours, cooled down to 190° C. The activated catalysts were used in the Fischer-Tropsch reaction with the following operating conditions: P=21 bar, GHSV=6,050H⁻¹.

### Effect of Cobalt Loading

### T=200° C.

| | | | CO₂ sel. | CH₄ sel. | C₅⁺ prod. |
|---|---|---|---|---|---|
| Catalyst | CO conv. (%) | C₅⁺ sel. (%) | (%) | (%) | (%) |
| Ex. 25 | 25 | 89 | 0.00 | 5.1 | 22 |
| Ex. 27 | 40 | 86 | 0.09 | 7.4 | 35 |
| Ex. 29 | 54 | 81 | 0.33 | 11 | 43 |

The CO conversion and the C₅⁺ productivity increase with the Co loading. The selectivity in CH₄ and CO₂ increases at the expense of the selectivity in C₅⁺.

### Effect of Addition of Ruthenium

### T=220° C.

| | | | CO₂ sel. | CH₄ sel. | C₅⁺ prod. |
|---|---|---|---|---|---|
| Catalyst | CO conv. (%) | C₅⁺ sel. (%) | (%) | (%) | (%) |
| Ex. 27 | 68 | 85 | 0.4 | 8.7 | 58 |
| Ex. 28 | 81 | 80 | 0.9 | 13 | 64 |

The CO conversion and the C₅⁺ productivity increase with the addition of ruthenium. The selectivity in CH₄ and CO₂ increases at the expense of the selectivity in C₅⁺.

The catalyst precursor produced according to Example 31 was activated in flowing hydrogen at GHSV of 8,000H⁻¹ at the heating rate of 1° C./min. to 400° C., and kept for 2 hours, cooled down to 160° C. The activated catalyst was used in the Fischer-Tropsch reaction with the following operating conditions: P=20 bar.

### Effect of GHSV

### T=199° C.

| | CO conv. | | CO₂ sel. | CH₄ sel. | C₅⁺ prod. |
|---|---|---|---|---|---|
| GHSV (H⁻¹) | (%) | C₅⁺ sel. (%) | (%) | (%) | (%) |
| 5,000 | 82 | 86 | 0.43 | 7.2 | 70 |
| 14,150 | 38 | 84 | 0.00 | 7.7 | 32 |

The CO conversion and the C₅⁺ productivity are divided by around 2 with the increase in GHSV (H⁻¹) from 5,000 to 14,150. The selectivities don't change with the GHSV.

### Effect of Temperature

### GHSV=5,000 H⁻¹

| | CO conv. | | CO₂ sel. | CH₄ sel. | C₅⁺ prod. |
|---|---|---|---|---|---|
| Temp. (° C.) | (%) | C₅⁺ sel. (%) | (%) | (%) | (%) |
| 173 | 28 | 87 | 0.02 | 4.0 | 25 |
| 180 | 39 | 86 | 0.17 | 4.7 | 33 |
| 191 | 66 | 87 | 0.02 | 5.7 | 58 |
| 199 | 82 | 86 | 0.43 | 7.2 | 70 |

The CO conversion and the C₅⁺ productivity increase with the increase in temperature. The C₅⁺ selectivity is constant. The selectivities in CO₂ and CH₄ increase with the temperature.

The catalyst precursor produced according to Example 29 was activated in flowing hydrogen at GHSV of 8,000 H⁻¹ at the heating rate of 1° C./min. to 400° C., and kept for 2 hours, cooled down to 160° C. The activated catalysts were used in the Fischer-Tropsch reaction with the following operating conditions: T=206° C., P=20 bar, GHSV=8,688 H⁻¹.

### Effect of Time on Stream

| Time on | CO conv. | | CO₂ sel. | CH₄ sel. | C₅⁺ prod. |
|---|---|---|---|---|---|
| stream (Hrs) | (%) | C₅⁺ sel. (%) | (%) | (%) | (%) |
| 46 | 66.60 | 82.95 | 0.24 | 9.91 | 55.24 |
| 70 | 65.55 | 83.36 | 0.24 | 9.70 | 54.64 |
| 94 | 64.60 | 83.22 | 0.22 | 9.51 | 53.76 |
| 119 | 62.99 | 82.88 | 0.16 | 9.57 | 52.21 |
| 143 | 62.57 | 82.82 | 0.18 | 9.45 | 51.82 |

The CO conversion and the C₅⁺ productivity decrease with the time on stream: the decrease of the conversion is around 1% per day. The C₅⁺, CO₂ and CH₄ selectivities are constant.

The catalyst synthesized in the presence of urea show robust performance over a wide range of GHSV, temperature, time on stream. The increase in Co loading and the addition of ruthenium increase the conversion without decreasing greatly the C₅⁺ selectivity. The addition of titanium also improves the selectivity in C₅⁺. These catalysts are suitable for application of the Fischer-Tropsch reaction at high GHSV (H⁻¹) and low temperature.

1. A precursor for a Fischer-Tropsch catalyst comprising:
(i) a catalyst support;
(ii) cobalt or iron on the catalyst support; and
(iii) one or more noble metals on the catalyst support,
wherein the cobalt or iron is at least partially in the form of its carbide in the as-prepared catalyst precursor.

2. The catalyst precursor of claim 1, wherein the catalyst support is a refractory solid oxide, carbon, a zeolite, boronitride, silicon carbide or a mixture of two or more thereof.

3. The catalyst precursor of claim 2, wherein the catalyst support is a refractory solid oxide which is alumina, silica, titania, zirconia, zinc oxide or a mixture of two or more thereof.

4. The catalyst precursor of claim 3, wherein the refractory solid oxide is silica, the primary catalyst metal is cobalt, and the surface of the silica is coated with a non-silicon oxide refractory solid oxide, the non-silicon oxide refractory solid oxide being zirconia, alumina or titania.

5. (canceled)

6. The catalyst precursor of claim 1, which comprises from 10 to 50% cobalt or iron (based on the weight of the metal as a percentage of the total weight of the catalyst precursor).

7. The catalyst precursor of claim 6, which comprises from 15 to 35% of cobalt or iron.

8-9. (canceled)

10. The catalyst precursor of claim 1, wherein the noble metal is one or more of Pd, Pt, Rh, Ru, Ir, Au, Ag or Os.

11. (canceled)

12. The catalyst precursor of claim 1, which comprises from 0.01 to 30% in total of noble metal(s) (based on the total weight of all noble metals present as a percentage of the total weight of the catalyst precursor).

13. The catalyst precursor of claim 12, which comprises from 0.05% to 20% in total of noble metal(s).

14-15. (canceled)

16. The catalyst precursor of claim 1, which includes one or more other metal-based components as promoters or modifiers.

17-18. (canceled)

19. The catalyst precursor of claim 7, which comprises from 0.1 to 10% in total of other metal(s) (based on the total weight of all the other metals as a percentage of the total weight of the catalyst precursor).

20. The catalyst precursor of claim 19, which comprises from 0.1 to 5% in total of other metals.

21. (canceled)

22. The catalyst precursor of claim 1, which contains from 0.0001 to 10% carbon (based on the weight of the carbon, in whatever form, in the catalyst as percentage of the total weight of the catalyst precursor).

23. The catalyst precursor of claim 22, which contains from 0.001 to 5% carbon.

24. (canceled)

25. A catalyst which is an activated catalyst precursor of claim 1.

26. Use of a catalyst of claim 25 in a Fischer-Tropsch process.

27. A method of preparing a catalyst precursor comprising:
depositing a solution or suspension comprising at least one catalyst metal precursor and a polar organic compound onto a catalyst support, wherein the solution or suspension contains little or no water;
if necessary, drying the catalyst support onto which the solution or suspension has been deposited; and
calcining the catalyst support onto which the solution or suspension has been deposited to convert at least part of said catalyst metal precursor to its carbide.

28-31. (canceled)

32. The method of claim 27, wherein, in the calcination step, the atmosphere contains little or no oxygen.

33-39. (canceled)

40. The method of claim 27, wherein the solution or suspension contains a cobalt-containing or an iron-containing precursor.

41-56. (canceled)

57. The method of claim 27, further including the step of activating the catalyst precursor to provide a catalyst.

58-66. (canceled)
